(19) United States
(12) Patent Application Publication
(10) Pub. No.: US 2005/0019907 A1
(43) Pub. Date: Jan. 27, 2005

(54) Obtaining normal disomic stem cells from chromosomally abnormal embryos

(76) Inventor: Santiago Munne, Hoboken, NJ (US)

Correspondence Address: Santiago Munne, 220 Washington Str. Apt 4, Hoboken, NJ, 07030, US

(21) Appl. No.: 10/625,100

(22) Filed: Jul. 22, 2003

(51) Int. Cl. C12N 5/08

(52) U.S. Cl. 435/366

(57) ABSTRACT

Product and Methods are described for obtaining normal disomic stem cells from chromosomally abnormal embryos.

### FEDERAL RESEARCH STATEMENT

[There were no federal funds used in this research]

### BACKGROUND OF INVENTION

Stem cells are derived from the inner cell mass (ICM) of the blastocyst, have a normal karyotype and can be maintained indefinitely in vitro culture. In contrast to human somatic stem cells, human embryos stem cells have the ability to develop to any cell type in the adult giving us a good source of material for further studies in tissue replacement, cancer therapy, drug discovery, among others.

Stem cells derived from human embryos have been established recently. Thomson in U.S. Pat. No. 5,843,780, titled “Primate embryonic stem cells”, issued Dec. 1, 1998 described for the first time the isolation of human embryonic stem cells lines. The use of normal embryos or the creation of human embryos for stem cell research has been controversial, and a ban on research with government funds has been applied in the US. There would be great utility, therefore, to use non-normal trisomic embryos as a source of chromosomally normal cells. This is particularly true, if the tissues become mosaic in culture with both trisomic and disomic normal cells. For instance, in prenatal diagnosis studies uniparental disomy has been extensively reported, and its origin explained as the loss through mosaicism of one chromosome in a trisomic cell. In trisomic embryos, the loss of the extra chromosome is known as trisomic zygote rescue.

Some cell lines can be both trisomic for some chromosomes, disomic for others and even tetrasomic for others. The 600MPE breast cancer cell line is disomic for the p-telomere-p22, trisomic for p22-centromere and tetrasomic for the q-arm of chromosome 1 (Pinkel, U.S. Pat. No. 6,335,167, Jan. 1, 2002, “Comparative genomic hybridization (CGH)”.

Morality of this Process

According to Dr. William Hurlbut, member of the President's Council on Bioethics, “a zygote differs fundamentally from an unfertilized egg, a sperm cel!, or later somatic cells; it possesses an inherent organismal unity and potency that such other cells lack. In biology, the whole (as the unified organismal principle of growth) precedes and produces the parts. It is this implicit whole, with its inherent potency, that endows the embryo with its human character and therefore its inviolable moral status. To interfere in its development is to transgress upon a life in process . . . . But what if we could to create an entity that lacks the qualities and capabilities essential to be designated a human life in process? could we truly create an artifact (a human creation for human ends) that is biologically and morally more akin to tissue or cell culture? . . . The intention in creating such an intrinsically limited “clonal artifact” would not be one of reproduction, but simply the desire to draw on natural organic potential through technological manipulation of biological materials. This intention is in keeping with the purposes of scientific research and medical therapy in which many “unnatural” manipulations are used for human benefit. In order to employ such an entity for research, it must be capable of yielding stem cells while lacking the capacity for the self-directed, integrated organic functioning that is essential for embryogenesis.

The intervention that precludes the possibility of human development would be undertaken at a stage before the development was initiated, and thus, no active potentiality, no human life in process, would be violated. If the created artifact were accorded a certain cautionary respect (as with all human tissues), even though not the full protection of human life, the consequences of such a program would not compromise any moral principle”.

Alternatively, embryos with chromosome abnormalities incompatible with birth (all trisomies with the exception of trisomy for gonosomes and chromosomes 13, 18, 21, as well as double trisomies involving any chromosome) do not have the potential to become a human life, and therefore should be considered not human embryos but products of failed conception, and therefore morally acceptable for stem cell production.

### SUMMARY OF INVENTION

This invention makes use of the novel observation that diploid cells under these novel culture conditions can be obtained from the extended culture of trisomic embryos cultured in monolayer and that these diploid cells are undifferentiated and contain stem cells. This invention consists, inter alia, of the use of trisomic embryonic cells which evolve some normal disomic cells and the use of these disomic normal cells to isolate stem cells.

### DETAILED DESCRIPTION

This invention has been enabled in a best mode using the following procedures.

Embryos classified by Preimplantation Genetic Diagnosis (PGD) as chromosomally abnormal were used for this study. On day 3 of development, each embryo had a single cell biopsied (Munn e et al., 2003) unless the nucleus could not be found after fixation, when a second cell was biopsied and fixed. For both the PGD analysis and the reanalysis, all cells were analyzed for chromosomes X, Y, 13, 15, 16, 17, 18, 21 and 22 using FISH protocols previously described by the inventor (Munn e et al. 1998). Based on these results, embryos classified as normal by PGD were replaced, while certain embryos classified as chromosomally abnormal were used for this study. Specifically, trisomic and monosomic embryos were used for this study. Institutional review board consents were signed for each patient.

Aneuploid embryos were cultured in sequential media until day 6. At that time, trophoectoderm of hatching blastocysts was biopsied using laser ablation following previously described protocols (Veiga et al., 1999) which is incorporated by reference. This step was performed in order to confirm that the embryo was chromosomally abnormal. The biopsied trophectoderm cells were fixed and analyzed by FISH following the same methods as used for PGD.

The remainder of the embryo was plated onto mouse embryonic fibroblast cells (ATCC-STO) previously mitotically inactivated by mitocimin C in gelatin-tissue culture dishes. Maintenance of mouse feeder cells was performed following commercial brand recommendations. The culture medium consisted in Dulbecco's modified eagle medium (DMEM without sodium pyruvate, glucose 4500 mgL-1; Life technologies) supplemented with 20% fetal bovine serum (FBS; life technologies), 0.1 mM à-mercaptoethanol, 1% non-essential amino acids, 1 mM L-glutamine, 50 units ml L-1 penicillin. At the time of embryo culture the medium was supplemented with human recombinant Leukemia Inhibitory factor (hLIF; Sigma) at 2000 units mL-1 and bFGF 4 ng/ml.

Embryos were cultured in this system until day 12, and then the human cells were fixed and analyzed by FISH following the above described method. In this initial study trophoectoderm and inner cell mass were not independently fixed for further FISH analysis. The different FISH results obtained on day 3, 6 and 12 were compared to determine if the PGD diagnosis was correct (day 3 vs day 6), and to determine if there was cell correction through extended culture (day 6 vs day 12).

44 embryos at blastocyst stage from aneuploid PGD embryos were biopsied at day 4 and plated onto mitotically mouse fibroblast cells. From those only 13 (29.5%) embryos attached to the feeder cells and 7/13 (53.8%) were fixed and analyzed by FSH at D12. All the embryos plated in culture showed TFE expansion and 1/7 (14.28%) embryos showed an ICM colony (embryo #1, Table 1).

The comparison of day 3 to day 6 indicates that of the 7 embryos with cells analyzed on day 12, one was a PGD misdiagnosis (embryo #1, table 1), which was mostly normal on day 6, another (#4, table 1) was a mostly normal embryo classified as normal/monosomy X by two-cell analysis during PGD Of the other 5 embryos, one was completely abnormal in both day 6 and day 12 (#6, table 1), while the others showed a progressive increase of normal cells, from 0-28% in day 6 to 21-80% on day 12. Interestingly, in culture all 7 embryos were mosaics by day 12.

Culture conditions may affect the chromosomal stability of cell lines. For instance the fusion of a mice and human cell produces hybrids that are known to lose human chromosomes at random until it stabilizes. The cleavage-stage is particularly prone to mosaicism in humans and also some mice strains (Munn é et al. 2002). Here we demonstrate that embryonic cells derived from chromosomally abnormal embryos can be a source of chromosomally normal cells that could be used to produce stem cells.

The observed reduction in trisomic cells cannot be due to the non-survival of trisomic embryos in culture, because they do survive to day 6 in sizable numbers when cultured as full embryos instead as in monolayer. Furthermore, the frequency of normal cells increases from day 6 (average of 30%) to day 12 (average of 50%). The most reasonable explanation for these observations is that trisomic cells revert to disomic cells, in extended culture, probably by losing a chromosome.

The yield is relatively low, only 7/44 chromosomally abnormal embryos developed in culture to day 12, and of those one was a PGD misdiagnosis. However, The PGD error rate is not 1/7 because these 7 embryos were those for which results on day 12 were obtained, and normal embryos develop better to blastocyst stage than abnormal ones.

It has been speculated that trisomic embryos can be corrected by three different ways: by anaphase-lag, non-disjunction and chromosome demolition. Anaphase-lag correction will result in one disomic and one trisomic daughter cell. Non-disjunction correction will result in one viable disomic and one lethal quadrisomic cell. In this case the number of cells will reduce delaying normal development. The third possibility is chromosome demolition correction, which consist in a deliberate fragmentation of one of the three chromosomes during metaphase or anaphase resulting in two disomic daughter cells. Confined placental mosaicism, in which placental tissue showed complete trisomy whereas the fetus was diploid, has been widely reported as a result of the loss of trisomic chromosomes in the embryonic tissue. To determine which of these three possibilities are the origin of the disomic cell lines observed in this study, fingerprinting experiments to determine if the same chromosome is always lost, or if there are different normal lines with different lost chromosomes, are pending.

Once the embryo is mosaic with disomic cell lines, the disomic cells may develop faster than the abnormal ones and differences in cleavage rate between disomic and aneuploid cell lines may result in an enrichment of the disomic line. For instance it has reported that aneuploid cell lines grow slower in extended culture than normal ones. The present method can be applied to obtain chromosomally normal stem cells from trisomic embryos. Because most trisomic embryos do not survive to term, those incompatible with life could be a more acceptable source of chromosomally normal stem cells than normal embryos.

TABLE 1
chromosome abnormalities found on the third, 6th and 12^{th} day of culture

| embryo | PGD-d3 | TFE-d6 | TFE-d12 | outcome |
|---|---|---|---|---|
| 1 | n = 1 | n = 50 | n = 44 | PGD misdiagnosis |
| | trisomy 21 | 76% normal | 61% normal | |
| | | 12% polyploid | 30% polyploid | |
| | | 12% aneuploidy 21 | 9% chaotic | |
| 2 | n = 1 | n = 54 | n = 117 | with self correction |
| | trisomy 13 | 74% trisomy 13 | 74% trisomy 13 | |
| | | 26% polyploid | 23% normal | |
| | | 0% normal | 3% chaotic | |
| 3 | n = 1 | n = 37 | n = 92 | with self correction |
| | trisomy 15 | 57% monosomy 22 | 16% monosomy 22 | |
| | monosomy 22 | 24% polyploid | 14% aneuploidy 15 | |
| | | 8% aneuploidy 15 | 40% monos 22 & pol | |
| | | 11% chaotic | 9% chaotic | |
| | | 0% normal | 21% normal | |
| 4 | n = 2 | n = 93 | n = 203 | Mosaic initially |
| | mosaic X/XY | 85% normal | 88% normal | mostly normal |
| | | 10% monosomy X | 8% polyploid | |
| | | 5% polyploid | 4% monosomy X | |
| 5 | n = 1 | n = 18 | n = 85 | with self correction |
| | haploid | 50% polyploid | 80% normal | |
| | | 22% normal | 7% polyploid | |
| | | 28% chaotic | 13% monosomy 15 | |
| 6 | n = 1 | n = 8 | n = 81 | without self correction |
| | chaotic | 37% monosomy X | 43% monosomy X | |
| | | 37% chaotic | 14% polyploid | |
| | | 26% polyploid | 43% chaotic | |
| 7 | n = 1 | n = 25 | n = 95 | 67% normal with |
| | mono 16 & 21 | 24% aneu 16, 21 | 28% normal | self correction |
| | | 24% polyploid | 15% polyploid | |
| | | 24% chaotic | 12% aneu 16, 22 | |
| | | | 6% chaotic | |

Method for the Derivation of Single-Cell Clones.

In order to produce a disomic cell line from a mixture of disomic and chromosomally abnormal cells, it is necessary to single-cell clone. Single-cell cloning can be done by methods well known to those of ordinary skill in the art and consisting of such steps as trypsinizing the stem cells and each individual cell is then plated in a different plate or other culture system. The yield is low (0.5 to 1%) and depends, as previously published, on culture conditions, being the best, those cultured in serum-free media supplemented with bFGF.

1. A disomic cell line derived from trisomic embryos

2. A stem cell line derived from said disomic cell lines of claim 1

3. A method of producing disomic cell lines consisting of the steps of:
a) culturing trisomic embryos onto mouse feeder cells consisting of mouse embryonic fibroblast cells (ATCC-STO) said mouse embryonic fibroblast cells having been previously mitotically inactivated by mitocimin C in gelatin-tissue culture dishes.
b) Maintaining said mouse feeder cells using Dulbecco's Modified Eagle Medium (DMEM) without sodium pyruvate, glucose 4500 mgL-1 supplemented with 20% fetal bovine serum, 0.1 mM à-mercaptoethanol, 1% non-essential amino acids, 1 mM L-glutamine, 50 units ml L-1 penicillin.
c) Supplementing said medium with human recombinant Leukemia Inhibitory factor at 2000 units mL-1 and bFGF 4 ng/ml
d) Culturing said embryos in said medium until day 12
e) Fixing and analyzing by FISH said embryonic cell lines Identifying and isolating disomic cell lines within said embryonic cell lines wherein disomic cell lines are produced.

4. A method of claim 3 wherein stem cell lines are isolated from said disomic cell lines.
